(19) United States
(12) Patent Application Publication
(10) Pub. No.: US 2024/0225726 A1
(43) Pub. Date: Jul. 11, 2024

(54) BASKET CATHETER HAVING INSULATED ABLATION ELECTRODES AND DIAGNOSTIC ELECTRODES

(71) Applicant: Biosense Webster (Israel) Ltd., Yokneam (IL)

(72) Inventors: Assaf GOVARI, Haifa (IL); Christopher Thomas BEECKLER, Brea, CA (US); Andres Claudio ALTMANN, Haifa (IL); Joseph Thomas KEYES, Sierra Madre, CA (US); Kevin Justin HERRERA, West Covina, CA (US)

(21) Appl. No.: 18/616,638

(22) Filed: Mar. 26, 2024

Related U.S. Application Data
(63) Continuation of application No. 17/028,913, filed on Sep. 22, 2020, now Pat. No. 11,950,841.

(51) Int. Cl. A61B 18/14; A61B 17/00; A61B 18/00; A61B 34/20

(52) U.S. Cl. CPC A61B 18/1492; A61B 34/20; A61B 2017/00526; A61B 2017/00867; A61B 2018/00077; A61B 2018/00083; A61B 2018/00267; A61B 2018/00351; A61B 2018/00577; A61B 2018/00613; A61B 2018/1467; A61B 2034/2051

(57) ABSTRACT

A catheter configured to be used in a medical procedure includes a shaft and electrically conductive splines. The splines can include an insulated portion and a non-insulated portion configured to electrically ablate tissue. The catheter can include one or more electrodes coupled to insulated portion(s) of the splines and configured to sense electrical signals in the tissue. The splines can be coupled to an apex, forming an expandable distal-end assembly. The catheter can include a bump stop coupled to the apex and configured to maintain a minimal distance between the apex and the distal tip of the shaft. An ablation system can include the catheter, a generator, and a processor configured to control ablation energy from the generator to the splines such that ablation energy is applied to a first spline without applying ablation energy to a second spline.

### CROSS-REFERENCE TO RELATED APPLICATIONS

This application is a continuation of prior filed U.S. patent application Ser. No. 17/028,913 filed on Sep. 22, 2020, which is related to U.S. patent application Ser. No. 17/028,907 filed on Sep. 22, 2020 and published as U.S. patent Pub. No. 2022/0087734 on Mar. 24, 2022, each of whose disclosures are incorporated herein by reference.

### FIELD OF THE INVENTION

The present invention relates generally to medical catheters, and particularly to ablation catheters having an expandable distal end.

### BACKGROUND OF THE INVENTION

Basket catheters may be used in various medical applications, such as in cardiology. Several types of basket catheters, having multiple splines, are designed to enable sensing and treating of arrhythmia.

For example, U.S. patent Application Publication 2014/0238175 describes methods, systems, and devices for providing treatment to a target site. The system may include a guide assembly, an expandable support device coupled with the distal end of the guide assembly, and an operative member disposed on the expandable support device. The expandable support device may be configured to transition between a collapsed and expanded configuration.

U.S. Pat. No. 6,292,695 describes a method of controlling cardiac fibrillation, tachycardia, or cardiac arrhythmia by the use of an electrophysiology catheter having a tip section that contains at least one stimulating electrode, the electrode being stably placed at a selected intravascular location. The electrode is connected to a stimulating means, and stimulation is applied across the wall of the vessel, transvascularly, to a sympathetic or parasympathetic nerve that innervates the heart at a strength sufficient to depolarize the nerve and effect the control of the heart.

U.S. Pat. No. 6,421,556 describes systems and methods for diagnosing and treating tissue transmit an electrical energy pulse that temporarily stuns a zone of tissue, temporarily rendering it electrically unresponsive. The systems and methods sense an electrophysiological effect. due to the transmitted pulse. The systems and methods alter an electrophysiological property of tissue in or near the zone based, at least in part, upon the sensed electrophysiological effect. The alteration of the electrophysiological property can be accomplished, for example, by tissue ablation or by the administration of medication. In an implementation, radio frequency energy is used to both temporarily stun tissue and to ablate tissue through a common electrode.

U.S. patent Application Publication 2015/0182282 describes a variety of systems and techniques for generating and applying plasmas and/or electric fields alone, or in combination with other therapies, to living tissue to treat different tissue conditions as well as other conditions, such as tumors, bacterial infections and the like while limiting electrical current generation within said tissue. U.S. patent Application. Publication No. 2006/0100669 shows and describe a system for atrial fibrillation using a basket catheter.

### SUMMARY OF THE INVENTION

An embodiment of the present invention that as described herein provides a catheter including a shaft for insertion into an organ of a patient, an expandable distal-end assembly, and one or more electrodes. The expandable distal-end assembly is coupled to the shaft and to an apex of the catheter, and includes multiple splines. In at least a given spline among the multiple splines, at least sixty (60) percent of a length of the given spline is non-insulated and is configured to make contact with tissue of the organ and to apply radiofrequency (RF) pulses to the tissue. The one or more electrodes are coupled to at least one of (i) an insulated section of one or more of the splines, and (ii) the apex, and, when placed in contact with the tissue, the one or more electrodes are configured to sense electrical signals in the tissue.

In some embodiments, the insulated section has at most forty (40) percent of the length of the given spline, and is positioned between the non-insulated length and the apex. In other embodiments, the insulated section is coated with an electrically-insulated layer. In yet other embodiments, at least the given spline includes nickel-titanium alloy.

In an embodiment, the apex has a flat surface, which is orthogonal to an axis of the shaft. In another embodiment, the catheter includes a processor, which is configured to produce an electrophysiological map of the tissue, indicative of the sensed electrical signals.

In some embodiments, first and second electrodes among the electrodes are disposed on the insulated section of the given spline, and the processor is configured to receive a bipolar signal sensed between the first and second electrodes. In other embodiments, the catheter includes third and fourth electrodes among the electrodes. The third electrode is disposed on an additional spline among the splines, and the fourth electrode is disposed on the apex. The processor is configured to receive at least one of: (i) a unipolar signal sensed between at least one of the electrodes and a reference electrode, and (ii) a bipolar signal sensed between any pair of the electrodes selected from among the first, second, third and fourth electrodes. In yet other embodiments, the catheter includes a bump stop, which is coupled to the apex and is configured to limit a minimal distance between the shaft and the apex.

There is additionally provided, in accordance with an embodiment of the present invention, a method for producing a catheter, the method includes receiving multiple splines. In at least a given spline among the multiple splines, at least sixty (60) percent of a length of the given spline is non-insulated for making contact with tissue of an organ of a patient and for applying radiofrequency (RF) pulses to the tissue, and at most forty (40) percent of the length of the given spline is insulated. The multiple splines are assembled together, such that the insulated length is positioned between the non-insulated length and an apex of the catheter for producing an expandable distal-end assembly. One or more electrodes are coupled to at least one of (i) the insulated length, and (ii) the apex, for sensing electrical signals when placed in contact with the tissue. The expandable distal-end assembly is coupled to a shaft of the catheter.

In some embodiments, the method includes coating the insulated length with an electrically-insulated layer.

There is further provided, in accordance with an embodiment of the present invention, a method including, inserting into an organ of a patient, a catheter having an expandable distal-end assembly that includes multiple splines, and in at least a given spline among the multiple splines, (i) at least sixty (60) percent of a length of the given spline is non-insulated for applying radiofrequency (RF) pulses to tissue of the organ, and (ii) at most forty (40) percent of the length of the given spline is insulated and is coupled to an apex of the catheter. At least one of the insulated length and the apex has one or more electrodes for sensing electrical signals in the tissue.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based position-tracking and ablation system, in accordance with an embodiment of the present invention;

FIGS. 2 and 3 are schematic, pictorial illustrations of distal-end assemblies in an expanded position, in accordance with embodiments of the present invention;

FIG. 4 is a flow chart that schematically illustrates a method for producing a basket catheter having insulated ablation electrodes, in accordance with an embodiment of the present invention; and

FIG. 5 is a flow chart that schematically illustrates a method for ablating tissue using the distal-end assembly of FIG. 3, in accordance with an embodiment of the present invention.

### DETAILED DESCRIPTION OF EMBODIMENTS

### Overview

Embodiments of the present invention that are described hereinbelow provide multiple configurations of improved distal-end assemblies used in a catheter of an ablation system. In some embodiments, the catheter comprises a shaft for inserting the distal-end assembly into a patient heart, and an expandable distal-end assembly, such as a basket, which is coupled to the shaft and to an apex of the catheter.

In some embodiments, the distal-end assembly comprises multiple splines, wherein at least one of, and typically all the splines are made from solid nitinol and have at least two sections. The first section of the splines is non-insulated, electrically conductive, and has at least sixty percent of the length of the splines. The first section is configured to make contact with tissue of the heart and to apply radiofrequency (RF) pulses to the tissue.

In some embodiments, the second section of the splines is insulated, comprises at most forty percent of the length of the splines, and positioned between the non-insulated section and the apex of the catheter. The insulated section may comprise the nitinol of the spline, which is coated with an electrically-insulated layer.

In some embodiments, the apex is electrically insulated from the spline and has a flat surface, which is orthogonal to an axis (e.g., longitudinal axis) of the shaft. Note that in the configuration described above, the first section is electrically conductive and is configured to ablate the heart tissue, whereas the second section and the apex are electrically insulated, and are configured to electrically insulate between the non-insulated sections of the splines. In such embodiments, in an ablation catheter having first and second splines, each of which having the first and second section, a processor of the ablation system is configured to control a radiofrequency (RF) pulse generator to apply one or more RF pulses to the first spline, without applying any RF pulses to the second spline.

Typically, prior to performing the ablation procedure, mapping of intra-cardiac signals is carried out, e.g., so as to define target tissue for ablation. In some embodiments, it is possible to use the non-insulated section of the splines for sensing the intra-cardiac signals. However, the non-insulated section, which comprises at least sixty percent of the spine, may have contact with a long section of the heart tissue, and therefore, may not be able to sense the intra-cardiac signals with sufficiently-high lateral resolution (e.g., about 1.0 mm) for mapping the tissue before ablation.

In some embodiments, the basket-shaped distal-end assembly described above, may comprise one or more diagnostic electrodes, also referred to herein as sensing electrodes. In the present example, the sensing electrodes are coupled to the electrically-insulated section and/or to the apex.

In some embodiments, one or more of the sensing electrodes are configured to sense unipolar signals, e.g., between the sensing electrode and a reference electrode attached to the skin or to any other tissue of the patient.

In other embodiments, two or more of the sensing electrodes are disposed on the apex and the insulated section and are configured to sense bipolar signals. For example, first and second sensing electrodes may be disposed on the insulated section of a first spline, a third sensing electrode may be disposed on the insulated section of a second spline, and a fourth sensing electrode may be disposed on the apex. In this configuration, the processor of the ablation system is configured to receive multiple bipolar signals from any pair from among the four sensing electrodes. For example, (i) a first bipolar signal between the first and second electrodes, (ii) a second bipolar signal between the second and third electrode, and (iii) a third bipolar signal between the third and fourth sensing electrodes.

The disclosed techniques are particularly effective for ablating (i) long and narrow tissue, e.g., when applying one or more RF pulses to a single spline, or (ii) long and wide tissue, e.g., when applying the RF pulses to multiple splines. Thus, the disclosed techniques may reduce the overall cycle time of an ablation procedure.

Moreover, the disclosed techniques reduce the need for attaching dedicated ablation electrodes to an expandable catheter, and therefore, improve the mechanical flexibility of the splines and reduce the costs associated with producing an expandable distal-end assembly of an ablation catheter.

### System Description

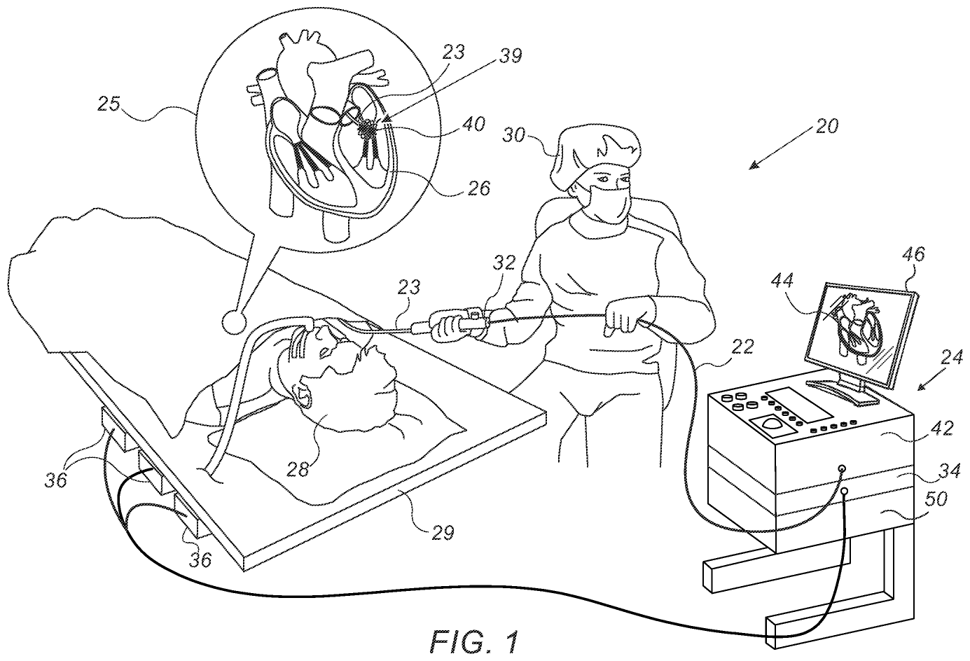

FIG. 1 is a schematic, pictorial illustration of a catheter-based position-tracking and ablation system 20, in accordance with an embodiment of the present invention. in some embodiments, system 20 comprises a catheter 22, in the present example an expandable cardiac catheter, and a control console 24. In the embodiment described herein, catheter 22 may be used for any suitable therapeutic and/or diagnostic purposes, such as ablation of tissue in a heart 26 and for mapping cardiac arrhythmias by sensing intra-cardiac electrical signals.

In some embodiments, console 24 comprises a processor 42, typically a general-purpose computer, with suitable front end and interface circuits for receiving signals from catheter 22 and for controlling other components of system 20 described herein. Processor 42 may be programmed in software to carry out the functions that are used by the system, and is configured to store data for the software in a memory 50. The software may be downloaded to console 24 in electronic form, over a network, for example, or it may be provided on non-transitory tangible media, such as optical, magnetic or electronic memory media. Alternatively, some or all of the functions of processor may be carried out using an application-specific integrated circuit (ASIC) or any suitable type of programmable digital hardware components.

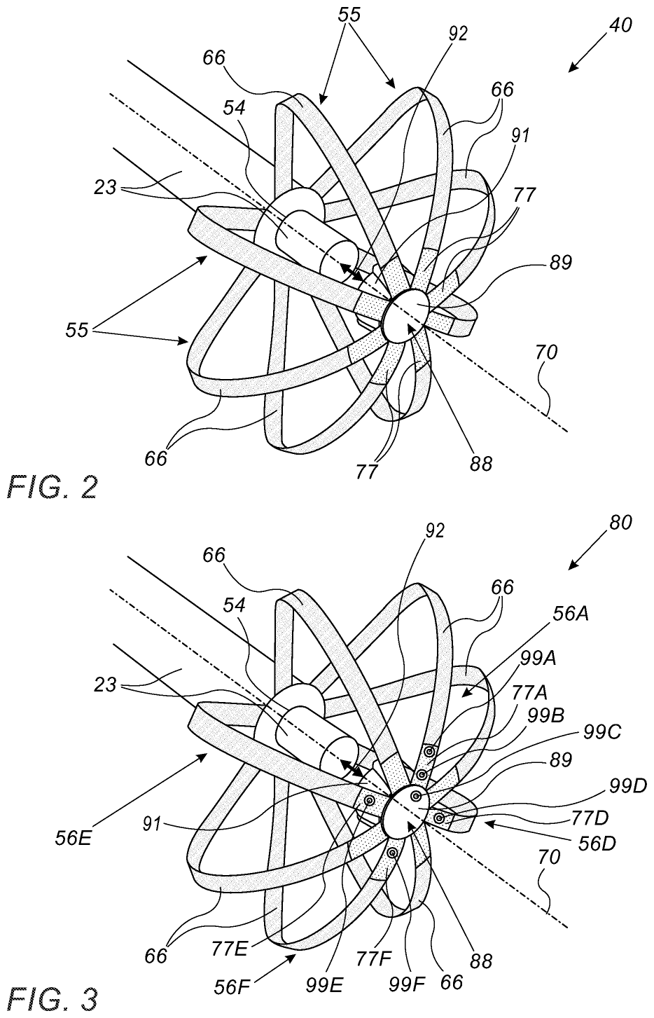

Reference is now made to an inset 25. In some embodiments, catheter 22 comprises a distal-end assembly 40 having multiple splines (shown in detail in FIGS. 2 and 3 below), and a shaft 23 for inserting distal-end assembly 40 to a target location for ablating tissue in heart 26. During an ablation procedure, physician 30 inserts catheter 22 through the vasculature system of a patient 28 lying on a table 29. Physician 30 moves distal-end assembly 40 to the target location in heart 26 using a manipulator 32 near a proximal end of catheter 22, which is connected to interface circuitry of processor 42.

In some embodiments, catheter 22 comprises a position sensor 39 of a position tracking system, which is coupled to the distal end of catheter 22, e.g., in close proximity to distal-end assembly 40. In the present example, position sensor 39 comprises a magnetic position sensor, but in other embodiments, any other suitable type of position sensor (e.g., other than magnetic-based) may be used.

Reference is now made back to the general view of FIG. 1. In some embodiments, during the navigation of distal-end assembly 40 in heart 26, processor 42 receives signals from magnetic position sensor 39 in response to magnetic fields from external field generators 36, for example, for the purpose of measuring the position of distal-end assembly 40 in heart 26. In some embodiments, console 24 comprises a driver circuit 34, configured to drive magnetic field generators 36. Magnetic field generators 36 are placed at known positions external to patient 28, e.g., below table 29.

In some embodiments, processor 42 is configured to display, e.g., on a display 46 of console 24, the tracked position of distal-end assembly 40 overlaid on an image 44 of heart 26.

The method of position sensing using external magnetic fields is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 1996005768, and in U.S. Patent Application Publications 2002/0065455 A1, 2003/0120150 A1 and 2004/0068178 A1, whose disclosures are all incorporated herein by reference.

### Distal-End Assembly Having Insulated Ablation Electrodes

FIG. 2 is a schematic, pictorial illustration of distal-end assembly 40 in expanded position, in accordance with an embodiment of the present invention.

In some embodiments, distal-end assembly 40 comprises multiple splines 55 made from solid nickel-titanium alloy, such as solid nitinol, or from any other suitable alloy or substance. Note that nitinol is selected for splines 55 for being electrically conductive and sufficiently flexible to conform to the tissue intended to be ablated. Thus, any other material selected for splines 55 has to be electrically conductive and sufficiently flexible to conform to the aforementioned tissue.

In some embodiments, at least a given spline 55, and typically each spline 55 of distal-end assembly 40, has an electrically-conductive section, referred to herein as a section 66, which is configured to serve as an ablation electrode for ablating tissue at the target location in heart 26.

In some embodiments, section 66 comprises at least sixty (60) percent of the length of spline 55. Section 66 is non-insulated (e.g., electrically conductive as described above) and is configured to make contact with tissue of heart 26, and to apply the radiofrequency (RF) pulses to the heart tissue during the ablation procedure.

In other embodiments, section 66 may comprise any other suitable portion of the length of spline 55.

In some embodiments, at least the aforementioned given spline 55, and typically each spline 55 of distal-end assembly 40, has a section 77, which is at most forty (40) percent of the length of the respective (e.g., given) spline 55. Section 77 is insulated and is positioned between section 66 and an apex 88 of catheter 22.

In some embodiments, section 77 is configured to electrically insulate between sections 66 of splines 55, at least at apex 88, which is typically electrically insulated from the spline. In other embodiments, apex 88 may comprise electrically conductive material(s) and may serve as an additional ablation electrode of distal-end assembly 40.

In some embodiments, apex 88 has a flat surface 89, which is typically (but not necessarily) orthogonal to an axis of shaft 23.

Note that section 66 has a typical length between about 10 mm and 40 mm. Therefore, when multiple splines 55 are applying pulses having predefined energy and duration, distal-end assembly 40 is configured to ablate an area of about 256 mm² in the tissue at the target location of heart 26.

In the context of the present disclosure and in the claims, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g., “about 90%” may refer to the range of values from 71% to 99%.

In some embodiments, distal-end assembly 40 comprises a coupling element, in the present example a ring 54, which is coupled to the proximal end of splines 55 and is configured to be threaded on shaft 23 for coupling between shaft 23 and distal-end assembly 40. Note that each spline 55 is electrically connected to catheter 22, e.g., via wired (not shown) extended from the proximal end of each spline 55. Thus, processor 42 may control the electrical connectivity between console 24 and each section 66 of splines 55. This controlled and selective connectivity is important for applying the RF pulses via selected splines 55 to the tissue of heart 26, and similarly, to receive intra-cardiac electrical signal from selected electrodes placed in contact with tissue of heart 26 (e.g., sections 66 or other electrodes described in FIG. 3 below).

In some embodiments, when physician 30 moves the distal end of catheter 22 to the target location in heart 26, distal-end assembly 40 is in a collapsed position with all splines 55 straightened. When distal-end assembly 40 is positioned at the target location in heart 26, physician 30 typically reduces the distance between ring 54 and apex 88 (e.g., by pulling apex 88 toward ring 54, or by pushing ring 54 toward apex 88, or using any other technique), so as to have distal-end assembly 40 at an expanded position with splines 55 bent as shown in FIG. 2.

In some embodiments, distal-end assembly 40 comprises a bump stop 91, which is coupled to apex 88 and is configured to control a minimal distance between shaft 23 and apex 88. At the expanded position shown in the example of FIG. 2, distal-end assembly 40 may have a gap distance 92 (measured along the longitudinal axis 70) between the tip of shaft 23 and bump stop 91. The gap distance 92 can be any value from about 1 mm to 13 mm depending on the overall diameter of the catheter. In one preferred embodiment, the gap distance 92 is about 3 mm as measured along the longitudinal axis 70. In such embodiments, when physician 30 further reduces the distance between ring 54 and apex 88, bump stop 91 is configured to make contact with shaft 23, so that gap distance 92 does not exist (i.e., equals zero), and thereby to limit the minimal distance between shaft 23 and apex 88. In other words, bump stop 91 serves as a hard stop, which is configured to prevent the basket distal-end assembly 40 from going completely flat, and thereby, buckling the nitinol base.

In some embodiments, physician 30 may use manipulator 32 for controlling the distance between apex 88 and ring 54, and therefore the amount of expansion of distal-end assembly 40. Note that, when applying the RF pulses to distal-end assembly 40, section 66 serves as an ablation electrode and applies the RF pulses to the tissue in contact therewith, whereas section 77 is insulated, and therefore, does not apply the RF pulses to the tissue. Moreover, sections 77 are electrically isolating between sections 66 of distal-end assembly 40. In this configuration, processor 42 may control the RF pulse generator to apply one or more RF pulses to a first section 66 of a first spline 55 and not to apply any RF pulses to a second section 66 of a second spline 55.

### Diagnostic Electrodes Coupled to the Apex of the Distal-End Assembly

FIG. 3 is a schematic, pictorial illustration of a distal-end assembly 80 in expanded position, in accordance with another embodiment of the present invention. Distal-end assembly 80 may replace, for example, distal-end assembly 40 of FIG. 1.

In some embodiments, distal-end assembly 80 comprises multiple splines 56, such as splines 56A, 56D, 56E, and 56F similar to splines 55 of FIG. 2 above and having insulated sections 77A, 77D, 77E, and 77F, respectively. Each spline 56 of distal-end assembly 80 comprises non-insulated section 66 having the dimensions, mechanical and electrical attributes described in FIG. 2 above.

In some embodiments, distal-end assembly 80 comprises one or more diagnostic electrodes, referred to herein as electrodes 99. In the present example, electrodes 99 comprise (i) electrodes 99A and 99B, coupled to section 77A, (ii) electrode 99C, coupled to surface 89 of apex 88, (iii) electrode 99D, coupled to section 77D, (iv) electrode 99E, coupled to section 77E, and (v) electrode 998, coupled to section 77F. In other embodiments, distal-end assembly 80 may comprise and other suitable number of electrodes 99 disposed on any sections 77 and/or apex 88 using any suitable configuration.

In some embodiments, when placed in contact with the tissue of heart. 26, electrodes 99 are configured to sense electrical signals in the tissue. In the context of the present disclosure and in the claims, the terms “sensing,” “mapping” and “diagnosing” are used interchangeably, wherein sensing electrodes (e.g., electrodes 99), which are configured for sensing the intra-cardiac signals, are producing signals used for mapping and diagnosing tissue in question of heart 26. In such embodiments, processor 42 is configured to produce, based on the intra-cardiac signals received from electrodes 99, an electrophysiological map of the tissue in question of heart 26.

In some embodiments, electrodes 99A and 99B are attached to section 77A of spline 56A, and are configured to sense a bipolar signal therebetween.

In some embodiments, a bipolar signal may be sensed between electrodes 99E and 99F, which are coupled to sections 77E and 77F of splines 56E and 56F, respectively. Additionally or alternatively, a bipolar signal may be sensed between electrode 99C that is coupled to surface 89, and electrode 99D that is coupled to section 77D of spline 56D. In other words, the bipolar signals may be sensed between (i) a pair of electrodes disposed on a single spline, and/or (ii) a pair of electrodes disposed on two different splines, and/or (iii) an electrode coupled to apex 88 and an electrode coupled to a spline, and/or (iv) a pair of electrodes (not shown) coupled to apex 88 at a predefined distance from one another, as shown for example on section 77A of spline 56A.

In other embodiments, at least one of electrodes 99 described above, is configured to sense a unipolar signal, for example relative to a reference electrode (not shown) placed in contact with tissue within the body of patient 28 or attached to the skin (not shown) of patient 28.

In some embodiments, electrodes 99 are coupled to respective sections 77 using any suitable technique, such as by crimping.

In some embodiments, distal-end assembly 80 may comprise electrical wires and/or electrical traces (neither shown), which are configured to electrically connect between each electrode 99 and processor 42. The electrical wires may be grouped in a braid running between console 24 and shaft 23, and at the distal end of shaft 23, each electrical wire is routed to a respective electrode 99.

In some embodiments, when distal-end assembly 80 is placed in contact with the tissue of heart 26, one or more of electrodes 99 are configured to generate potential gradient signals in response to sensed electrical potentials in the tissue of heart 26. Position sensor 39, which is fixed at the distal end of catheter 22 at a known distance from each electrode 99, is configured to generate position signals in response to the sensed external magnetic fields.

In some embodiments, based on (i) the position signal received from position sensor 39 and (ii) the signals received from one or more electrodes 99, processor 42 is configured to display, e.g., on display 46, an electrophysiological map of the electrical potentials sensed by electrodes 99 at respective locations in the tissue of heart 26.

In some embodiments, based on the electrophysiological mapping of the tissue of heart 26, physician 30 may position selected splines 56 in contact with the tissue to-be-ablated in heart 26. Subsequently, physician 30 and/or processor 42 may control system 20 to apply the RF pulses, so as to ablate the tissue of heart 26.

This particular configuration of system 20 and distal-end assemblies 40 and 80 are shown by way of example, in order to illustrate certain problems that are addressed by embodiments of the present invention and to demonstrate the application of these embodiments in enhancing the performance of such an ablation system. Embodiments of the present invention, however, are by no means limited to this specific sort of example system, and the principles described herein may similarly be applied to other sorts of medical systems, such as but not limited to electrosurgical systems and irreversible electroporation (IRE) ablation systems.

### Producing Basket-Shaped Distal-End Assembly

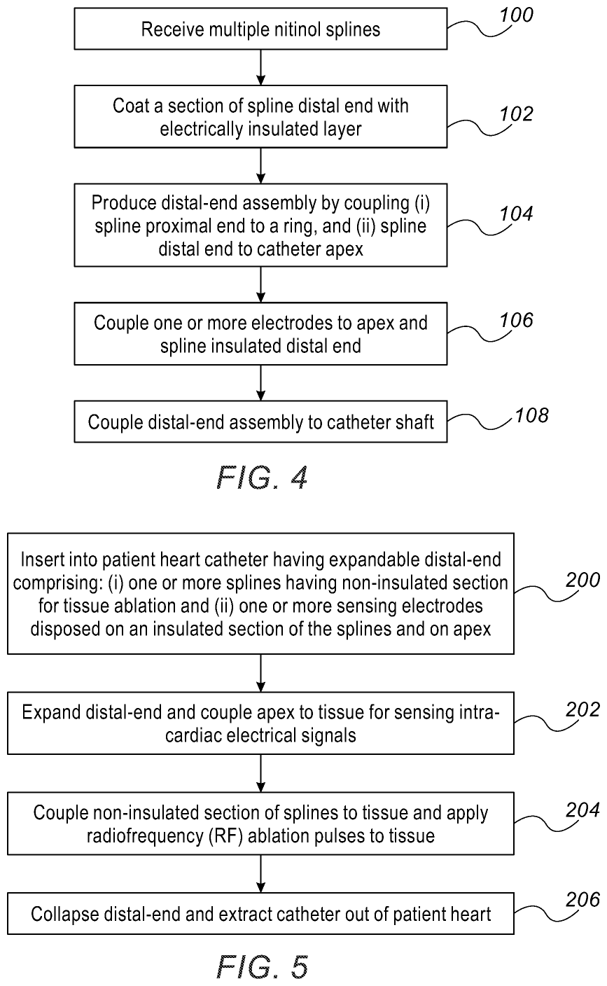

FIG. 4 is a flow chart that schematically illustrates a method for producing distal-end assemblies 40 and 80, accordance with an embodiment of the present invention. The method begins at a spline receiving step 100, with receiving multiple splines 55 made from nitinol or from any suitable nickel-titanium alloy, or from any other alloy suitable to serve as an ablation electrode as described in FIGS. 2A and 2B above.

At a coating step 102, section. 77 of at least one of, and typically all splines 55 is coated with an electrically-insulated layer. At a distal-end assembly producing step 104, distal-end assemblies 40 and 80 are produced by coupling (i) the proximal end of all splines 55 to ring 54, and (ii) sections 77 located at the distal end of all splines 55 to catheter apex 88.

In other embodiments, one or more of splines 55 may already be coated with the aforementioned electrically-insulated layer at spline receiving step 100, so that step 102 may be redundant, and therefore, be excluded from the method.

At an electrodes coupling step 106, which is carried out only in the production of distal-end assembly 80, one or more electrodes 99 are coupled to apex 88 and/or to section 77 of one or more splines 55, and are connected to electrical wires for conducting signals between one or more (typically all) electrodes 99 and processor 42, as described in FIG. 2B above. Note that step 106 is not carried out in the production of distal-end assembly 40.

At a distal-end assembly coupling step 108, which concludes the method, distal-end assembly 40 or distal-end assembly 80 is coupled to shaft 23 along axis 70, as described in FIGS. 2A and 2B above. Note that in the production of distal-end assembly 40, after performing step 104, the method proceeds directly to step 108 without performing step 106, which is carried out only in the production of distal-end assembly 80.

In summary, steps 100, 102, 104 and 108 are used for producing distal-end assembly 40, and all steps 100, 102, 104, 106 and 108 are used for producing distal-end assembly 80.

This particular method of FIG. 4 for producing distal-end assemblies 40 and 80 is provided by way of example, in order to illustrate certain problems that are addressed by embodiments of the present invention and to demonstrate the application of these embodiments in producing distal-end assemblies 40 and 80 for enhancing the performance of system 20. Embodiments of the present invention, however, are by no means limited to this specific sort of example production processes, and the principles described herein may similarly be applied to other sorts of distal-end assemblies used in system 20 or in other sorts of medical systems, such as but not limited to electrosurgical systems and irreversible electroporation (IRE) ablation systems.

### Performing Ablation Procedure Using the Basket-Shaped Distal-End Assembly

FIG. 5 is a flow chart that schematically illustrates a method for ablating tissue using distal-end assembly 80, in accordance with an embodiment of the present invention. The method begins at a catheter insertion step 200, with inserting distal-end assembly, which is described in detail in FIG. 3 below, into heart 26. In essence, one or more splines 56 of distal-end assembly 80 have: (i) section 66 configured for ablating the tissue of heart 26, and (ii) one or more electrodes, such as electrodes 99A-99F shown in FIG. 3 above, configured for sensing the intra-cardiac electrical signals in the tissue of heart 26.

At a sensing step 202, after placing distal-end assembly 80 in close proximity to tissue at the target location in heart 26, physician 30 controls the expansion of distal-end assembly 80 to the expanded position. The expansion may be carried out, for example, by reducing the distance between ring 54 and apex 88. Note that the minimal distance between ring 54 and apex 88 is limited by bump stop 91, as described in FIG. 1 above. In some embodiments, at sensing step 202, at least one of and typically both apex 88 and sections 77 (e.g., sections 77A, 77D, 77E and 77F) are coupled to the tissue for sensing the intra-cardiac electrical signals.

At an ablation step 204, based on the signals sensed in step 202, physician 30 manipulates distal-end assembly 80 and couples section 66 of one or more splines 56 (e.g., splines 56A, 56D, 56E and 56F) to the tissue for performing RF ablation. Note that in step 202 distal-end assembly 80 is positioned such that axis 70 is approximately orthogonal to the tissue for placing surface 89 of apex 88 in contact with the tissue for sensing. Moreover, in step 204, distal-end assembly 80 is positioned such that axis is approximately parallel to the tissue, so that one or more sections 66 are placed in contact with, and ablating the tissue.

At a catheter extraction step 206 that terminates the method, after concluding the ablation, physician 30 controls catheter 22 to collapse distal-end assembly 80 (e.g., by increasing the distance between ring 54 and apex 88), and subsequently, extracts catheter 22 out of the body of patient 28.

In other embodiments, processor 42 may hold the electrophysiological mapping of the tissue, before the ablation procedure or at least before step 202. In such embodiments, ablation step 204 is carried out based on the mapping after catheter insertion step 200, and sensing step 202 may be carried out after step 204, so as to check whether applying additional RF pulses is required after step 204 for concluding the ablation procedure. In case applying additional RF pulses is required, the method loops back to ablation step 204 followed by sensing step 202 for one or more iterations until concluding the ablation.

In alternative embodiments, sensing step 202 may be carried out before and after ablation step 204, so as to sense the intra-cardiac electrical signals for controlling the ablation process as described above.

Although the embodiments described herein manly address basket catheters, the techniques described herein can also be used in any other suitable medical probes having splines for applying energy to tissue.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

What is claimed is:

1. A catheter configured to be used in a medical procedure, the catheter comprising:
a shaft extending along a longitudinal axis and comprising a distal tip;
a plurality of electrically conductive splines coupled to the distal tip, each spline of the electrically conductive splines comprising an insulated portion and a non-insulated portion, the non-insulated portion being configured to make contact with a tissue of an anatomical structure and electrically ablate the tissue; and
one or more electrodes coupled to the insulated portion of one or more splines of the plurality of electrically conductive splines, each of the one or more electrodes being configured to sense electrical signals in the tissue.

2. The catheter of claim 1, further comprising:
an apex disposed at a distal end of the catheter, wherein the plurality of electrically conductive splines are coupled to the apex and forming an expandable distal-end assembly between the distal tip of the shaft and the apex.

3. The catheter of claim 2, wherein the apex comprises a non-insulated portion configured to make contact with the tissue and electrically ablate the tissue.

4. The catheter of claim 2,
wherein the apex comprises an insulated portion, and
wherein an electrode of the one or more electrodes is coupled to the insulated portion of the apex.

5. The catheter of claim 2, further comprising:
a bump stop coupled to a proximal side of the apex and separated from the distal tip by a collapsible gap.

6. The catheter of claim 5, wherein the bump stop is configured to contact the distal tip of the shaft to maintain a minimal distance between the apex and the distal tip of the shaft when a force is applied to the expandable distal-end assembly.

7. The catheter of claim 5, wherein the collapsible gap comprises a gap distance of about 3 mm as measured along the longitudinal axis when the expandable distal-end assembly is in an expanded position without force applied to the expandable distal-end assembly.

8. The catheter of claim 1, wherein at least 60 percent of a length at least one spline is non-insulated and is configured to make contact with tissue of the anatomical structure and to electrically ablate the tissue.

9. The catheter of claim 1, wherein the plurality of electrically conductive splines are configured to ablate an area of about 256 mm² of the tissue.

10. The catheter of claim 1, wherein the non-insulated portion of each spline of the plurality of electrically conductive splines measures between about 10 mm and about 40 mm.

11. The catheter of claim 1,
wherein the shaft is configured to be manipulated to position the plurality of electrically conductive splines within the anatomical structure,
wherein the anatomical structure comprises a heart of a patient,
wherein the non-insulated portions of the plurality of electrically conductive splines are configured to deliver radiofrequency and/or irreversible electroporation pulses to electrically ablate tissue of the heart.

12. A catheter configured to be used in a medical procedure, the catheter comprising:
a shaft extending along a longitudinal axis and comprising a distal tip;
an apex disposed at a distal end of the catheter;
a plurality of electrically conductive splines coupled to the apex and the distal tip of the shaft and forming an expandable distal-end assembly between the distal tip of the shaft and the apex; and
a bump stop coupled to the apex and configured to contact the distal tip of the shaft to maintain a minimum distance between the apex and the distal tip of the shaft when a force is applied to the expandable distal-end assembly.

13. The catheter of claim 12,
wherein each spline of the electrically conductive splines comprises an insulated portion and a non-insulated portion, and
wherein the non-insulated portion is configured to make contact with a tissue of an anatomical structure and electrically ablate the tissue.

14. The catheter of claim 13, further comprising:
one or more electrodes coupled to the insulated portion of one or more splines of the plurality of electrically conductive splines, each of the one or more electrodes being configured to sense electrical signals in the tissue.

15. The catheter of claim 13,
wherein the apex comprises an electrically conductive material, and
wherein at least a portion of the apex is non-insulated and configured to make contact with a tissue of an anatomical structure and electrically ablate the tissue.

16. The catheter of claim 15, further comprising:
one or more electrodes coupled to the insulated portion of the apex, each of the one or more electrodes being configured to sense electrical signals in the tissue.

17. An ablation system comprising:
a catheter comprising a shaft and a plurality of electrically conductive splines coupled to a distal end of the shaft;
a generator; and
a processor configured to control ablation energy provided by the generator to the plurality of electrically conductive splines such that ablation energy is applied to a first spline of the plurality of electrically conductive splines without applying ablation energy to a second spline of the plurality of electrically conductive splines.

18. The ablation system of claim 17,
wherein each spline of the electrically conductive splines comprises an insulated portion and a non-insulated portion, the non-insulated portion being configured to make contact with a tissue of an anatomical structure and electrically ablate the tissue, and
wherein the catheter further comprises one or more electrodes coupled to the insulated portion of one or more splines of the plurality of electrically conductive splines, each of the one or more electrodes being configured to sense electrical signals in the tissue.

19. The ablation system of claim 17,
wherein the catheter further comprises an apex, and
wherein the plurality of electrically conductive spines are coupled to the apex to form an expandable distal-end assembly between a distal tip of the shaft and the apex.

20. The ablation system of claim 19,
wherein the catheter comprises a bump stop coupled to the apex and configured to contact the distal tip of the shaft to maintain a minimum distance between the apex and the distal tip of the shaft when a force is applied to the expandable distal-end assembly.
